(19) United States
(12) Patent Application Publication
(10) Pub. No.: US 2005/0137181 A1
(43) Pub. Date: Jun. 23, 2005

(54) Method for treatment of reactive arthritis or bursitis

(76) Inventors: Ernest L. Bonner, Hillsborough, CA (US); Robert Hines, Fayetteville, NC (US)

Correspondence Address: BEESON SKINNER BEVERLY LLP, ONE KAISER PLAZA, SUITE 2360, OAKLAND, CA, 94612, US

(21) Appl. No.: 11/054,921

(22) Filed: Feb. 9, 2005

Related U.S. Application Data
(63) Continuation-in-part of application No. 10/896,612, filed on Jul. 20, 2004.
Application No. 10/896,612 is a continuation of application No. 10/271,117, filed on Oct. 15, 2002, now Pat. No. 6,765,000.
Application No. 10/271,117 is a continuation of application No. 09/510,704, filed on Feb. 22, 2000, now Pat. No. 6,465,473.
Application No. 09/510,704 is a continuation-in-part of application No. 09/270,962, filed on Mar. 17, 1999, now Pat. No. 6,087,382.

(51) Int. Cl. A61K 31/65; A61K 31/522; A61K 31/4172

(52) U.S. Cl. 514/192; 514/263.32; 514/400

(57) ABSTRACT

A method for treatment for conditions in human beings associated with either or both reactive arthritis or bursitis comprising administering a combination of a member of the family of synthetic purine nucleoside analog antiviral drugs, a member of the tetracycline family, and a member of the nitroimidazole family, or alternatively, administering a combination of a member of the family of synthetic purine nucleoside analog antiviral drugs, a member of the beta-lactam family, and a member of the nitroimidazole family, or alternatively, administering a combination of a member of the family of synthetic purine nucleoside analog antiviral drugs, a member of the macrolide antimicrobial family, and a member of the nitroimidazole family, or alternatively, administering a combination of a member of the family of synthetic purine nucleoside analog antiviral drugs, a member of the ketolide antimicrobial family, and a member of the nitroimidazole family.

This is a continuation-in-part of application Ser. No. 10/896,612 filed Jul. 20, 2004, still pending, which was a continuation of application Ser. No. 10/271,117 filed Oct. 15, 2002, now U.S. Pat. No. 6,765,000, which was a continuation of application Ser. No. 09/510,704, filed Feb. 22, 2000, now U.S. Pat. No. 6,465,473, which was a continuation-in-part of application Ser. No. 09/270,962, filed Mar. 17, 1999, now U.S. Pat. No. 6,087,382.

### BACKGROUND OF THE INVENTION

This invention relates to an improved method for treatment of symptoms associated in humans with reactive arthritis or idiopathic bursitis.

Reactive arthritis refers to a spondyloarthritity which usually arises as a complication of an infection elsewhere in the body. Reactive arthritis can be caused by species of Shigella bacteria (most notably Shigella flexneri), Yersinia enterocolitica, Campylobacter jejuni, several species of Salmonella, genitourinary pathogens, Chlamydia trachomatis, Neisseria gonorrhea, Ureaplasma urealyticum, Streptococcus pyogenes, and other yet unidentified infectious agents.

Reactive arthritis commonly occurs in young men and women, but can occur at any age. Sufferers experience joint pain, stiffness, redness or swelling. Common symptoms may include fatigue, malaise, fever, and weight loss. The joints of the lower extremities, including the knee, ankle, and joints of the foot, are the most common sites of involvement, but symptoms can also occur in the wrists, fingers, elbows, shoulders, neck, and lower back. Other symptoms may include urethritis and prostatitis in males, and cervicitis or salpingitis in females. Ocular disease is common ranging from transient, asymptomatic conjunctivitis to aggressive anterior uveitis that occasionally results in blindness. Mucocutaneous lesions and nail changes are frequent. On less frequent or rare occasions manifestations of reactive arthritis include cardiac conduction defects, aortic insufficiency, central or peripheral nervous system lesions, and pleuropulmonary infiltrates.

Treatment of patients suffering from reactive arthritis with nonsteroidal anti-inflammatory drugs (“NSAIDs”) provides some benefit, although symptoms of reactive arthritis are rarely completely alleviated and some patients fail to respond at all. The preferred initial treatment of choice for acute reactive arthritis is indomethacin in divided doses of 75 to 150 milligrams per day. The NSAID of last resort is phenylbutazone, in doses of 100 milligrams twice or three times per day, because of its potentially serious side effects. Patients with debilitating symptoms refractory to NSAID therapy may be treated with cytotoxic agents such as azathioprine or methotrexate, or with sulfasalazine. Tendinitis, other lesions, and uveitis may benefit from corticosteroids. Minocycline hydrochloride, a semisynthetic derivative of tetracycline, is indicated for infections caused by at least Shigella microorganisms, Streptococcus pyogenes, and Neisserie gonorrhoeae. It is therefore an accepted treatment in incidents of reactive arthritis triggered by these biological entities.

Long-term follow-up studies have suggested that some joint symptoms persist in many, if not most, patients with reactive arthritis. Recurrences of the more acute symptoms are common and as many as twenty-five percent of patients either become unable to work or are forced to change occupations because of persistent joint problems.

Bursitis is inflammation of a bursa, a thin-walled sac lined with synovial tissue. The function of the bursa is to facilitate movement of tendons and muscles over bony prominences. Bursitis may be caused by excessive frictional forces, trauma, systemic disease such as rheumatoid arthritis or gout, or infection. The most common form of bursitis is subacromial. Trochanteric bursitis causes patients to experience pain over the lateral aspect of the hip and upper thigh, and tenderness over the posterior aspect of the greater trochanter. Retrocalcaneal bursitis involves the bursa located between the calcaneus and the posterior surface of the Achilles tendon. Pain is experienced at the back of the heel, and swelling appears on either or both of the medial and lateral sides of the tendon. Retrocalcaneal bursitis occurs in association with spondyloarthritities, rheumatoid arthritis, gout, and trauma.

Treatment of bursitis generally consists of prevention of the aggravating condition, rest of the involved part, an NSAID, and local steroid injection. In the long term, bursitis can result in loss of use of a joint and chronic pain syndrome.

The long term effects of reactive arthritis and bursitis range from chronic pain to crippling disability. It is also thought that many instances of osteoarthritis and psoriatic arthritis are in actuality reactive arthritis. Unfortunately, current procedures for management treat the symptoms of these diseases rather than their underlying pathogens.

### SUMMARY OF THE INVENTION

Significant benefits can be obtained by treating humans affected with conditions associated with reactive arthritis or bursitis using combinations of acyclovir, minocycline hydrochloride, and metronidazole or, alternatively, valacyclovir hydrochloride, minocycline hydrochloride, and metronidazole.

Acyclovir and valacyclovir hydrochloride are members of the family of synthetic purine nucleoside analog antiviral drugs. The chemical name of acyclovir is 2-amino-1,9-dihydro-9-[(2-hydroxyethoxy)methyl]-6H-purin-6-one. Acyclovir is commercially available under the brand name ZOVIRAX® in capsules, tablets, or suspension. Acyclovir has demonstrated anti-viral activity against herpes simplex virus types I and II, varicella-zoster virus, Epstein-Barr virus and cytomegalovirus, both in vitro and in vivo.

Valacyclovir hydrochloride (sold under the brand name Valtrex®) is the hydrochloride salt of L-valyl ester of acyclovir. The chemical name of valacyclovir hydrochloride is L-valine 2-[(2-amino-1,6-dihydro-6-oxo-9H-purin-9-yl) methoxy]ethyl ester, monohydrochloride. Valacyclovir hydrochloride is rapidly and nearly completely converted to acyclovir in the body.

Minocycline hydrochloride is a member of the tetracycline family. Minocycline hydrochloride is a bacteriostatic antibiotic which exerts its antimicrobial effect by inhibition of bacterial protein synthesis. It has been shown to be effective against gram-negative bacteria, some gram-positive bacteria and other microorganisms.

Metronidazole is a member of the nitroimidazole family. Metronidazole is an oral synthetic antiprotozoal and antibacterial agent. Heretofore it has been indicated for treatment of symptomatic trichomoniasis, intestinal amebiasis, and a wide range of intra-abdominal, skin, gynecological, bone and joint, lower respiratory tract and central nervous system infections, bacterial septicemia and endocarditis.

One embodiment of a combination for treatment of the symptoms in human beings of reactive arthritis or idiopathic bursitis, or both, comprises the combination of acyclovir, minocycline hydrochloride, and metronidazole. An alternative combination comprises the substitution of valacyclovir hydrochloride in place of acyclovir. The pharmaceutical dosages of the compounds of the combination may be administered in capsules, tablets, in suspension form, or by injection.

The invention provides a method for administration of a pharmaceutical combination that puts the diseases of reactive arthritis and bursitis into remission. Treatment with the combination may effect a cure of reactive arthritis and bursitis, but definitive testing has not been performed to confirm that fact.

It is therefore a primary object of the invention to provide a method for administration of a combination for treating conditions in human beings associated with either or both reactive arthritis or idiopathic bursitis.

Another object of the invention is to provide a method for administration of a treatment for conditions in human beings associated with either or both reactive arthritis or idiopathic bursitis that puts the disease being treated into full remission.

A further object of the invention is to provide a method for administration of a treatment for any constellation of symptoms amenable to treatment using the above combination, including for example, cases of reactive arthritis which have been misdiagnosed as osteoarthritis or psoriatic arthritis.

### DETAILED DESCRIPTION OF THE INVENTION

U.S. Pat. No. 6,087,382 to Bonner, Jr., et al., describes a method of treatment involving administration of a combination of L-lysine, minocycline hydrochloride, and metronidazole. An alternate method includes administration of InH for those individuals who have tested positively for mycobacterial exposure, along with the underlying combination of L-lysine, minocycline hydrochloride, and metronidazole. Another method described in U.S. Pat. No. 6,465,473 to Bonner, Jr., et al., includes administration of valacyclovir hydrochloride with the underlying combination of L-lysine, minocycline hydrochloride, and metronidazole. A third method of treatment, described in applicants' U.S. Pat. No. 6,197,776, includes administration of acyclovir with the underlying combination of L-lysine, minocycline hydrochloride, and metronidazole. An embodiment of the treatment, described in applicants' U.S. Pat. No. 6,765,000, comprises a pharmaceutical combination including acyclovir, minocycline. hydrochloride, and metronidazole. Alternatively, the treatment may include valacyclovir hydrochloride, minocycline hydrochloride, and metronidazole. Either of these embodiments may be supplemented with administration of pyridoxine hydrochloride, glucosamine, manganese, vitamin C, and desalinated seawater, such as Essence of Life.

Administration will generally be accomplished orally via capsules, tablets, or in suspension form, but delivery could be accomplished by injection, or any other method commonly used for administration of internal medicines.

Like L-lysine, acyclovir inhibits herpes simplex viruses, but by a different mechanism. While L-lysine tends to stop the virus from replicating by inhibiting the initiation of the replication process, acyclovir inhibits effective replication of actively replicating viral particles, e.g., by stopping replication of herpes viral DNA. This is accomplished by either competitive inhibition or inactivation of viral DNA polymerase or incorporation and termination of the growing viral DNA chain. It is believed that acyclovir results in a substantial benefit due to its inhibition of virus replication. In double-blind testing, it has been found that the administration of the combination of acyclovir, minocycline hydrochloride, and metronidazole is an effective treatment for reactive arthritis or bursitis. Acyclovir has never been used in the prior art for treatment of arthritis or bursitis. It does not appear to be effective alone for the treatment of these diseases. The preferred dose of acyclovir is 400 mg twice daily. The daily dose of acyclovir may vary from 200 mg to 4 grams.

The preferred dose of valacyclovir hydrochloride is 500 mg twice daily. The total daily dose of valacyclovir hydrochloride may vary from 125 mg to 4 grams.

The preferred dose of minocycline hydrochloride is an initial dosage of 200 mg followed by doses of 100 mg twice per day. Daily doses of minocycline hydrochloride following the initial administration of 200 mg may vary from 50 mg to 200 mg. Based upon their similar properties, it is expected that other members of the tetracycline family such as doxycycline can be effectively substituted, in the combination, for minocycline hydrochloride.

The preferred dose of metronidazole is 250-500 mg twice per day. The total dose per day of metronidazole may vary from 100 mg to 1,000 mg.

It is known that the combination of minocycline hydrochloride, InH, and metronidazole inhibits the multiplication of susceptible organisms, including Shigella, Salmonella, Chlamydia, Streptococci, and mycobacteria. Applicants have also determined that the combination of L-lysine, minocycline hydrochloride, and metronidazole provides a medically effective treatment for reactive arthritis and bursitis. See U.S. Pat. No. 6,087,382. It has also been shown that the combination of acyclovir, L-lysine, minocycline hydrochloride, and metronidazole provides an effective treatment for these conditions. See U.S. Pat. No. 6,197,776. Individuals with severe symptoms, including joint swelling and joint contractures, who were not thought to be candidates for treatment using the combination of L-lysine, minocycline hydrochloride, and metronidazole only, have also experienced substantial beneficial effects in response to treatment with that combination and valacyclovir hydrochloride.

The preferred embodiment of the present invention comprises the combination of acyclovir, or its prodrug valacyclovir hydrochloride, with minocycline hydrochloride and metronidazole. An alternate embodiment comprises valacyclovir hydrochloride with minocycline hydrochloride and metronidazole. These combinations each provide a medically effective treatment for reactive arthritis and bursitis. The total combination of medicines in each of these embodiments presents a broad spectrum approach that it is believed effectively addresses the underlying pathogenesis for reactive arthritis and what has previously been referred to as idiopathic bursitis, and further is a beneficial treatment for reactive arthritis in particular cases wherein the symptom complex has been misdiagnosed as osteoarthritis or psoriatic arthritis, or in any other similar cases of misdiagnosis.

### EXAMPLES

The following examples serve to illustrate the invention, but is not meant to restrict its effective scope.

### Example 1

A 77 year old female presented with complaints of neck, upper back, lower back, bilateral shoulder, bilateral wrist, digits of hands, bilateral hip, and bilateral ankle pains of years duration. The patient complained of associated stiffness in those same joints. Her physical examination was remarkable for tenderness at her neck, right shoulder, elbow bilaterally, wrist bilaterally, the metacarpal phalangeal and the proximal interphalangeal joints of her right hand, hip bilaterally, knee bilaterally, and the Achilles insertion area bilaterally. The Sed rate and rheumatoid factors were normal. This patient was diagnosed with reactive arthritis and was started on a treatment consisting of 125 mg of metronidazole, 250 mg of valacyclovir hydrochloride, and 50 mg of minocycline hydrochloride twice daily. After 69 days of such treatment, the patient noted pain in the palm of her left hand only. She further denied any stiffness. Physical examination on the 69^{th} day did not reveal any tenderness. Thus, treatment effected resolution of pain, stiffness, and tenderness in this patient.

### Example 2

A 52 year old male presented with complaints of bilateral knee and left wrist pain. He also noted associated morning stiffness. He was treated with minocyline hydrochloride 100 mg BID and acyclovir 400 mg BID. This resulted in significant improvement, but not total resolution of his complaints of pain and stiffness in his knees and left wrist.

### Example 3

A 45 year old male with multiple joint pain and associated stiffness who was treated with metronidazole 250 BID and minocycline 100 mg BID experienced a decrease in pain severity and a slight decrease in stiffness with such treatment.

Amoxicillin is a member of the beta-lactam family of anti-microbial agents. Amoxicillin is a semi-synthetic antibacterial agent in the beta-lactam family with a broad spectrum of bactericidal activity. Beta-lactams are widely considered to be a medically acceptable alternative to tetracyclines. The preferred dose of amoxicillin is 500 mg twice per day. The total dose per day of amoxicillin may vary from 50 mg to 3 gm.

Azithromycin is a member of the macrolide family of anti-microbial agents. Azithromycin acts by interfering with microbial protein synthesis and has a wide spectrum of activity against microorganisms. Members of the macrolide family are widely considered to be a medically acceptable alternative to tetracyclines. The preferred dose of azithromycin is 125 mg twice per day. The total dose per day of azithromycin may vary from 50 mg to 2 gm.

Telithromycin is a member of the ketolide family of anti-microbial agents. Telithromycin is a semi-synthetic antibacterial agent which acts by blocking protein synthesis. Members of the ketolide family are widely considered to be a medically acceptable alternative to tetracyclines. The preferred dose of telithromycin is 400 mg twice per day. The total dose per day of telithromycin may vary from 50 mg to 2 gm.

There have been thus described certain preferred embodiments of a pharmaceutical formulation for treatment of conditions in human beings associated with either or both reactive arthritis or idiopathic bursitis. While preferred embodiments have been described and disclosed, it will be recognized by those with skill in the art that modifications are within the true scope and spirit of the invention. The appended claims are intended to cover all such modifications.

1. A method for medically treating the symptoms of reactive arthritis or bursitis in humans comprising:
administering to a patient an effective amount of the combination of a member of the family of synthetic purine nucleoside analog antiviral drugs or a pharmaceutically acceptable ester or a metabolite thereof, a member of the beta-lactam family or a metabolite thereof, and a member of the nitroimidazole family or a metabolite thereof.

2. The method for treatment of claim 1 wherein:
said member of the family of synthetic purine nucleoside analog antiviral drugs comprises acyclovir.

3. The method for treatment of claim 1 wherein:
said member of the family of synthetic purine nucleoside analog antiviral drugs comprises valacyclovir hydrochloride.

4. The method for treatment of claim 1 wherein:
said member of the beta-lactam family comprises amoxicillin.

5. The method for treatment of claim 1 wherein:
said member of the nitroimidazole family comprises metronidazole.

6. The method for treatment of claim 1 wherein:
said member of the family of synthetic purine nucleoside analog antiviral drugs comprises acyclovir or a pharmaceutically acceptable ester thereof, said member of the beta-lactam family comprises amoxicillin, and said member of the nitroimidazole family comprises metronidazole.

7. The treatment of claim 6 wherein:
between approximately 50 mg to 3 gm of amoxicillin are administered.

8. The treatment of claim 6 wherein:
between approximately 100 mg to 4 gm of acyclovir are administered.

9. The treatment of claim 6 wherein:
between approximately 100 mg to 1 gm of metronidazole are administered.

10. The treatment of claim 6 wherein:
between approximately 100 mg to 4 gm of acyclovir are administered, between approximately 50 mg to 3 gm of amoxicillin are administered, and between approximately 100 mg to 1 gm of metronidazole are administered.

11. The treatment of claim 6 wherein:
approximately 400 mg of acyclovir are administered twice daily.

12. The treatment of claim 6 wherein:
approximately 500 mg of amoxicillin are administered twice daily.

13. The treatment of claim 6 wherein:
approximately 250 mg of metronidazole are administered twice daily.

14. The treatment of claim 6 wherein:
approximately 400 mg of acyclovir are administered twice daily, approximately 500 mg of amoxicillin are administered twice daily, and approximately 250 mg of metronidazole are administered twice daily.

15. The method for treatment of claim 1 wherein:
said member of the family of synthetic purine nucleoside analog antiviral drugs comprises valacyclovir or a pharmaceutically acceptable ester thereof, said member of the beta-lactam family comprises amoxicillin, and said member of the nitroimidazole family comprises metronidazole.

16. The treatment of claim 15 wherein:
between approximately 125 mg to 4 gm of valacyclovir hydrochloride are administered daily.

17. The treatment of claim 15 wherein:
between approximately 50 mg to 3 gm of amoxicillin are administered daily.

18. The treatment of claim 15 wherein:
between approximately 100 mg to 1 gm of metronidazole are administered daily.

19. The treatment of claim 15 wherein:
between approximately 125 mg to 4 gm of valacyclovir hydrochloride are administered daily, between approximately 50 mg to 3 gm of amoxicillin are administered daily, and between approximately 100 mg to 1 gm of metronidazole are administered daily.

20. The treatment of claim 15 wherein:
approximately 500 mg of valacyclovir hydrochloride are administered twice daily.

21. The treatment of claim 15 wherein:
approximately 500 mg of amoxicillin are administered twice daily.

22. The treatment of claim 15 wherein:
approximately 250 mg of metronidazole are administered twice daily.

23. The treatment of claim 15 wherein:
approximately 500 mg of valacyclovir hydrochloride are administered twice daily, approximately 500 mg of amoxicillin are administered twice daily, and approximately 250 mg of metronidazole are administered twice daily.

24. The method for treatment of claim 1 wherein:
said member of the family of synthetic purine nucleoside analog antiviral drugs comprises acyclovir or a pharmaceutically acceptable ester thereof, said member of the beta-lactam family comprises a fluoroquinolone, and said member of the nitroimidazole family comprises metronidazole.

25. The treatment of claim 24 wherein:
said pharmaceutically acceptable ester of acyclovir is valacyclovir hydrochloride.

26. The treatment of claim 25 wherein:
between approximately 125 mg to 4 gm of valacyclovir hydrochloride are administered daily.

27. The treatment of claim 25 wherein:
between approximately 125 mg to 4 gm of valacyclovir hydrochloride are administered, between approximately 50 mg to 3 gm of amoxicillin are administered, and between approximately 100 mg to 1 gm of metronidazole are administered.

28. The treatment of claim 25 wherein:
approximately 500 mg of valacyclovir hydrochloride are administered twice daily.

29. A method for medically treating the symptoms of reactive arthritis or bursitis in humans comprising:
administering to a patient an effective amount of the combination of a member of the family of synthetic purine nucleoside analog antiviral drugs or a pharmaceutically acceptable ester or a metabolite thereof, a member of the macrolide antimicrobial family or a metabolite thereof, and a member of the nitroimidazole family or a metabolite thereof.

30. The method for treatment of claim 29 wherein:
said member of the family of synthetic purine nucleoside analog antiviral drugs comprises acyclovir.

31. The method for treatment of claim 29 wherein:
said member of the family of synthetic purine nucleoside analog antiviral drugs comprises valacyclovir hydrochloride.

32. The method for treatment of claim 29 wherein:
said member of the macrolide antimicrobial family comprises azithromycin.

33. The method for treatment of claim 29 wherein:
said member of the nitroimidazole family comprises metronidazole.

34. The method for treatment of claim 29 wherein:
said member of the family of synthetic purine nucleoside analog antiviral drugs comprises acyclovir or a pharmaceutically acceptable ester thereof, said member of the macrolide antimicrobial family comprises azithromycin, and said member of the nitroimidazole family comprises metronidazole.

35. The treatment of claim 34 wherein:
between approximately 50 mg to 2 gm of azithromycin are administered.

36. The treatment of claim 34 wherein:
between approximately 100 mg to 4 gm of acyclovir are administered.

37. The treatment of claim 34 wherein:
between approximately 100 mg to 1 gm of metronidazole are administered.

38. The treatment of claim 34 wherein:
between approximately 100 mg to 4 gm of acyclovir are administered, between approximately 50 mg to 2 gm of azithromycin are administered, and between approximately 100 mg to 1 gm of metronidazole are administered.

39. The treatment of claim 34 wherein:
approximately 400 mg of acyclovir are administered twice daily.

40. The treatment of claim 34 wherein:
approximately 125 mg of azithromycin are administered twice daily.

41. The treatment of claim 34 wherein:
approximately 250 mg of metronidazole are administered twice daily.

42. The treatment of claim 34 wherein:
approximately 400 mg of acyclovir are administered twice daily, approximately 125 mg of azithromycin are administered twice daily, and approximately 250 mg of metronidazole are administered twice daily.

43. The method for treatment of claim 29 wherein:
said member of the family of synthetic purine nucleoside analog antiviral drugs comprises valacyclovir or a pharmaceutically acceptable ester thereof, said member of the macrolide antimicrobial family comprises azithromycin, and said member of the nitroimidazole family comprises metronidazole.

44. The treatment of claim 43 wherein:
between approximately 125 mg to 4 gm of valacyclovir hydrochloride are administered daily.

45. The treatment of claim 43 wherein:
between approximately 50 mg to 2 gm of azithromycin are administered daily.

46. The treatment of claim 43 wherein:
between approximately 100 mg to 1 gm of metronidazole are administered daily.

47. The treatment of claim 43 wherein:
between approximately 125 mg to 4 gm of valacyclovir hydrochloride are administered daily, between approximately 50 mg to 2 gm of azithromycin are administered daily, and between approximately 100 mg to 1 gm of metronidazole are administered daily.

48. The treatment of claim 43 wherein:
approximately 500 mg of valacyclovir hydrochloride are administered twice daily.

49. The treatment of claim 43 wherein:
approximately 125 mg of azithromycin are administered twice daily.

50. The treatment of claim 43 wherein:
approximately 250 mg of metronidazole are administered twice daily.

51. The treatment of claim 43 wherein:
approximately 500 mg of valacyclovir hydrochloride are administered twice daily, approximately 125 mg of azithromycin are administered twice daily, and approximately 250 mg of metronidazole are administered twice daily.

52. The method for treatment of claim 29 wherein:
said member of the family of synthetic purine nucleoside analog antiviral drugs comprises acyclovir or a pharmaceutically acceptable ester thereof, said member of the macrolide antimicrobial family comprises a fluoroquinolone, and said member of the nitroimidazole family comprises metronidazole.

53. The treatment of claim 52 wherein:
said pharmaceutically acceptable ester of acyclovir is valacyclovir hydrochloride.

54. The treatment of claim 53 wherein:
between approximately 125 mg to 4 gm of valacyclovir hydrochloride are administered daily.

55. The treatment of claim 53 wherein:
between approximately 125 mg to 4 gm of valacyclovir hydrochloride are administered, between approximately 50 mg to 2 gm of azithromycin are administered, and between approximately 100 mg to 1 gm of metronidazole are administered.

56. The treatment of claim 53 wherein:
approximately 500 mg of valacyclovir hydrochloride are administered twice daily.

57. A method for medically treating the symptoms of reactive arthritis or bursitis in humans comprising:
administering to a patient an effective amount of the combination of a member of the family of synthetic purine nucleoside analog antiviral drugs or a pharmaceutically acceptable ester or a metabolite thereof, and a member of the macrolide antimicrobial family or a metabolite thereof.

58. The method for treatment of claim 57 wherein:
said member of the family of synthetic purine nucleoside analog antiviral drugs comprises acyclovir.

59. The treatment of claim 58 wherein:
between approximately 100 mg to 4 gm of acyclovir are administered.

60. The treatment of claim 59 wherein:
approximately 400 mg of acyclovir are administered twice daily.

61. The method for treatment of claim 57 wherein:
said member of the family of synthetic purine nucleoside analog antiviral drugs comprises valacyclovir hydrochloride.

62. The treatment of claim 61 wherein:
between approximately 125 mg to 4 gm of valacyclovir hydrochloride are administered daily.

63. The treatment of claim 62 wherein:
approximately 500 mg of valacyclovir hydrochloride are administered twice daily.

64. The method for treatment of claim 57 wherein:
said member of the macrolide antimicrobial family comprises azithromycin.

65. The treatment of claim 64 wherein:
between approximately 50 mg to 2 gm of azithromycin are administered.

66. The treatment of claim 65 wherein:
approximately 125 mg of azithromycin are administered twice daily.

67. A method for medically treating the symptoms of reactive arthritis or bursitis in humans comprising:
administering to a patient an effective amount of the combination of a member of the family of synthetic purine nucleoside analog antiviral drugs or a pharmaceutically acceptable ester or a metabolite thereof, and a member of the beta-lactam family or a metabolite thereof.

68. The method for treatment of claim 67 wherein:
said member of the family of synthetic purine nucleoside analog antiviral drugs comprises acyclovir.

69. The treatment of claim 68 wherein:
between approximately 100 mg to 4 gm of acyclovir are administered.

70. The treatment of claim 69 wherein:
approximately 400 mg of acyclovir are administered twice daily.

71. The method for treatment of claim 67 wherein:
said member of the family of synthetic purine nucleoside analog antiviral drugs comprises valacyclovir hydrochloride.

72. The treatment of claim 71 wherein:
between approximately 125 mg to 4 gm of valacyclovir hydrochloride are administered daily.

73. The treatment of claim 72 wherein:
approximately 500 mg of valacyclovir hydrochloride are administered twice daily.

74. The method for treatment of claim 67 wherein:
said member of the beta-lactam family comprises amoxicillin.

75. The treatment of claim 74 wherein:
between approximately 50 mg to 3 gm of amoxicillin are administered.

76. The treatment of claim 75 wherein:
approximately 500 mg of amoxicillin are administered twice daily.

77. A method for medically treating the symptoms of reactive arthritis or bursitis in humans comprising:
administering to a patient an effective amount of the combination of a member of the family of synthetic purine nucleoside analog antiviral drugs or a pharmaceutically acceptable ester or a metabolite thereof, a member of the ketolide antimicrobial family or a metabolite thereof, and a member of the nitroimidazole family or a metabolite thereof.

78. The method for treatment of claim 77 wherein:
said member of the family of synthetic purine nucleoside analog antiviral drugs comprises acyclovir.

79. The method for treatment of claim 77 wherein:
said member of the family of synthetic purine nucleoside analog antiviral drugs comprises valacyclovir hydrochloride.

80. The method for treatment of claim 77 wherein:
said member of the ketolide antimicrobial family comprises telithromycin.

81. The method for treatment of claim 77 wherein:
said member of the nitroimidazole family comprises metronidazole.

82. The method for treatment of claim 77 wherein:
said member of the family of synthetic purine nucleoside analog antiviral drugs comprises acyclovir or a pharmaceutically acceptable ester thereof, said member of the ketolide antimicrobial family comprises telithromycin, and said member of the nitroimidazole family comprises metronidazole.

83. The treatment of claim 82 wherein:
between approximately 50 mg to 2 gm of telithromycin are administered.

84. The treatment of claim 82 wherein:
between approximately 100 mg to 4 gm of acyclovir are administered.

85. The treatment of claim 82 wherein:
between approximately 100 mg to 1 gm of metronidazole are administered.

86. The treatment of claim 82 wherein:
between approximately 100 mg to 4 gm of acyclovir are administered, between approximately 50 mg to 2 gm of telithromycin are administered, and between approximately 100 mg to 1 gm of metronidazole are administered.

87. The treatment of claim 82 wherein:
approximately 400 mg of acyclovir are administered twice daily.

88. The treatment of claim 82 wherein:
approximately 400 mg of telithromycin are administered twice daily.

89. The treatment of claim 82 wherein:
approximately 250 mg of metronidazole are administered twice daily.

90. The treatment of claim 82 wherein:
approximately 400 mg of acyclovir are administered twice daily, approximately 400 mg of telithromycin are administered twice daily, and approximately 250 mg of metronidazole are administered twice daily.

91. The method for treatment of claim 77 wherein:
said member of the family of synthetic purine nucleoside analog antiviral drugs comprises valacyclovir or a pharmaceutically acceptable ester thereof, said member of the ketolide antimicrobial family comprises telithromycin, and said member of the nitroimidazole family comprises metronidazole.

92. The treatment of claim 91 wherein:
between approximately 125 mg to 4 gm of valacyclovir hydrochloride are administered daily.

93. The treatment of claim 91 wherein:
between approximately 50 mg to 2 gm of telithromycin are administered daily.

94. The treatment of claim 91 wherein:
between approximately 100 mg to 1 gm of metronidazole are administered daily.

95. The treatment of claim 91 wherein:
between approximately 125 mg to 4 gm of valacyclovir hydrochloride are administered daily, between approximately 50 mg to 2 gm of telithromycin are administered daily, and between approximately 100 mg to 1 gm of metronidazole are administered daily.

96. The treatment of claim 91 wherein:
approximately 500 mg of valacyclovir hydrochloride are administered twice daily.

97. The treatment of claim 91 wherein:
approximately 400 mg of telithromycin are administered twice daily.

98. The treatment of claim 91 wherein:
approximately 250 mg of metronidazole are administered twice daily.

99. The treatment of claim 91 wherein:
approximately 500 mg of valacyclovir hydrochloride are administered twice daily, approximately 400 mg of telithromycin are administered twice daily, and approximately 250 mg of metronidazole are administered twice daily.

100. The method for treatment of claim 77 wherein:
said member of the family of synthetic purine nucleoside analog antiviral drugs comprises acyclovir or a pharmaceutically acceptable ester thereof, said member of the ketolide antimicrobial family comprises a fluoroquinolone, and said member of the nitroimidazole family comprises metronidazole.

101. The treatment of claim 82 wherein:
said pharmaceutically acceptable ester of acyclovir is valacyclovir hydrochloride.

102. The treatment of claim 101 wherein:
between approximately 125 mg to 4 gm of valacyclovir hydrochloride are administered daily.

103. The treatment of claim 101 wherein:
between approximately 125 mg to 4 gm of valacyclovir hydrochloride are administered, between approximately 50 mg to 2 gm of telithromycin are administered, and between approximately 100 mg to 1 gm of metronidazole are administered.

104. The treatment of claim 101 wherein:
approximately 500 mg of valacyclovir hydrochloride are administered twice daily.

105. A method for medically treating the symptoms of reactive arthritis or bursitis in humans comprising:
administering to a patient an effective amount of the combination of a member of the family of synthetic purine nucleoside analog antiviral drugs or a pharmaceutically acceptable ester or a metabolite thereof, and a member of the ketolide antimicrobial family or a metabolite thereof.

106. The method for treatment of claim 105 wherein:
said member of the family of synthetic purine nucleoside analog antiviral drugs comprises acyclovir.

107. The treatment of claim 106 wherein:
between approximately 100 mg to 4 gm of acyclovir are administered.

108. The treatment of claim 107 wherein:
approximately 400 mg of acyclovir are administered twice daily.

109. The method for treatment of claim 105 wherein:
said member of the family of synthetic purine nucleoside analog antiviral drugs comprises valacyclovir hydrochloride.

110. The treatment of claim 109 wherein:
between approximately 125 mg to 4 gm of valacyclovir hydrochloride are administered daily.

111. The treatment of claim 110 wherein:
approximately 500 mg of valacyclovir hydrochloride are administered twice daily.

112. The method for treatment of claim 105 wherein:
said member of the ketolide antimicrobial family comprises telithromycin.

113. The treatment of claim 112 wherein:
between approximately 50 mg to 2 gm of telithromycin are administered.

114. The treatment of claim 113 wherein:
approximately 400 mg of telithromycin are administered twice daily.

115. A method for medically treating the symptoms of reactive arthritis or bursitis in humans comprising:
administering to a patient an effective amount of the combination of a member of the nitroimidazole family or a metabolite thereof and a member of the ketolide antimicrobial family or a metabolite thereof.

116. The method for treatment of claim 115 wherein:
said member of the nitroimidazole family comprises metronidazole.

117. The treatment of claim 116 wherein:
between approximately 100 mg to 1 gm of metronidazole are administered.

118. The treatment of claim 117 wherein:
approximately 250 mg of metronidazole are administered twice daily.

119. The method for treatment of claim 115 wherein:
said member of the ketolide antimicrobial family comprises telithromycin.

120. The treatment of claim 119 wherein:
between approximately 50 mg to 2 gm of telithromycin are administered.

121. The treatment of claim 120 wherein:
approximately 400 mg of telithromycin are administered twice daily.

122. A method for medically treating the symptoms of reactive arthritis or bursitis in humans comprising:
administering to a patient an effective amount of the combination of a member of the nitroimidazole family or a metabolite thereof and a member of the beta-lactam family or a metabolite thereof.

123. The method for treatment of claim 122 wherein:
said member of the nitroimidazole family comprises metronidazole.

124. The treatment of claim 123 wherein:
between approximately 100 mg to 1 gm of metronidazole are administered daily.

125. The treatment of claim 124 wherein:
approximately 250 mg of metronidazole are administered twice daily.

126. The method for treatment of claim 122 wherein:
said member of the beta-lactam family comprises amoxicillin.

127. The treatment of claim 126 wherein:
between approximately 50 mg to 3 gm of amoxicillin are administered.

128. The treatment of claim 127 wherein:
approximately 500 mg of amoxicillin are administered twice daily.

129. A method for medically treating the symptoms of reactive arthritis or bursitis in humans comprising:
administering to a patient an effective amount of the combination of a member of the nitroimidazole family or a metabolite thereof and a member of the beta-lactam family or a metabolite thereof.

130. The method for treatment of claim 129 wherein:
said member of the nitroimidazole family comprises metronidazole.

131. The treatment of claim 130 wherein:
between approximately 100 mg to 1 gm of metronidazole are administered daily.

132. The treatment of claim 131 wherein:
approximately 250 mg of metronidazole are administered twice daily.

133. The method for treatment of claim 129 wherein:
said member of the beta-lactam family comprises amoxicillin.

134. The treatment of claim 133 wherein:
between approximately 50 mg to 3 gm of amoxicillin are administered.

135. The treatment of claim 134 wherein:
approximately 500 mg of amoxicillin are administered twice daily.
